(19) United States
(12) Patent Application Publication
(10) Pub. No.: US 2025/0091286 A1
(43) Pub. Date: Mar. 20, 2025

(54) IN VIVO 3D BIOPRINTING DEVICE AND METHOD

(71) Applicant: THE REGENTS OF THE UNIVERSITY OF CALIFORNIA, Oakland, CA (US)

(72) Inventors: David Barnes BERRY, La Jolla, CA (US); Shaochen CHEN, San Diego, CA (US)

(21) Appl. No.: 18/959,285

(22) Filed: Nov. 25, 2024

Related U.S. Application Data
(63) Continuation of application No. PCT/US2023/023763, filed on May 26, 2023.
(60) Provisional application No. 63/346,807, filed on May 27, 2022.

(51) Int. Cl. B29C 64/106; B29C 64/209; B29C 64/264; B29C 64/321; B33Y 10/00; B33Y 40/00; B33Y 70/10; B33Y 80/00; C09D 11/04

(52) U.S. Cl. CPC B29C 64/106; B29C 64/209; B29C 64/264; B29C 64/321; B33Y 10/00; B33Y 70/10; C09D 11/04; B29K 2995/0056; B33Y 40/00; B33Y 80/00

(57) ABSTRACT

A device for in vivo 3D bioprinting includes an elongated hollow tube configured that can be inserted into a living body. A feed tube within the hollow tube conveys a liquid polymerizable biomaterial to an extrusion nozzle positioned at the target site. A light guide within the hollow tube conducts polymerizing light from a light source to polymerize the biomaterial that has been extruded at the target site. Arthroscopic procedures employing the device enable in vivo attachment of tissue to bone or other tissue, or replacement of loss tissue or bone volume.

### RELATED APPLICATIONS

This is a continuation under 35 U.S.C. 365 (c) of International Application No. PCT/US2023/023763, filed May 26, 2023, which claims the benefit of the priority of U.S. Provisional Application No. 63/346,807, filed May 27, 2022, each of which is incorporated herein by reference in its entirety.

### FIELD OF THE INVENTION

The present invention relates to a device and method for printing of biomaterials in vivo, and more particularly to a device and method for 3-dimensional arthroscopic bioprinting.

### BACKGROUND

Three-dimensional (3D) printing technology, an advanced additive manufacturing technology, has been demonstrated for fabrication of custom-designed or complex structures with wide medical applications. Bioprinting, i.e., use of bioink containing cells to 3D print living obstacles such as tissue or organ, has great potential in advancing medicine, especially in regenerative medicine. Currently, commonly used 3D bioprinting systems include inkjet printing, extrusion printing, light-assisted printing, and laser direct writing. Currently, the in vivo application strategies for 3D-printed macroscale products are limited to surgical implantation or in situ 3D printing at the exposed trauma, both requiring exposure of the application site. However, a major goal of clinical treatments involves the use of minimally invasive or noninvasive approaches. For internal injuries under the skin, surgery exposing trauma can damage surrounding tissues, leading to secondary injury. Meanwhile, for plastic surgery, noninvasive methods are highly desirable to reduce scarring. Such objectives are not achievable using existing 3D printing technologies, motivating efforts to develop noninvasive 3D printing technologies that can noninvasively fabricate tissue-covered bioink into customized products, including living tissue constructs in situ.

Digital light processing (DLP)-based 3D bioprinting technology, a light-assisted bioprinting method, has attracted much attention in recent decades for its high cell viability of post-printing and superior printing speed and resolution. Systems for DLP-based bioprinting are known and have been described in a number of publications. See, for example, P. Wang, et al., “Controlled Growth Factor Release in 3D-Printed Hydrogels”, Adv. Healthcare Mater. 2019, 1900977, and J. Koffler, et al., “Biomimetic 3D-printed scaffolds for spinal cord injury repair”, Nature Medicine, 25 (2), February 2019, each of which is incorporated herein by reference. Currently, DLP-based 3D printing has been demonstrated for use in multiple-tissue reconstruction or repair, including spinal cord, peripheral nerve, and blood vessel injury. Conventionally, ultraviolet (UV) or blue light (wavelength ˜380 nm-˜410 nm) is exploited to assist bioprinting via photopolymerization. However, it is difficult to use UV or blue light as a tool for noninvasive manufacturing because of its poor tissue-penetration ability. Near-infrared (NIR) light can penetrate into deep tissue and has been used for controlled drug release, photodynamic therapy, photothermal therapy, in vivo imaging, 3D image visualization, and optogenetics in vivo. Moreover, similar to UV or blue light, NIR light has potential to initiate photopolymerization. Use of NIR-induced photopolymerization has been reported for transdermal 3D printing by Y. Chen, et al., in “Noninvasive in vivo 3D bioprinting”, Science Advances, Vol. 6, No. 23 5 Jun. 2020, and A. Urciulo, et al, “Intravital three-dimensional bioprinting”, Nature Biomed. Eng., 4, 901-915 (2020), which are incorporated herein by reference. While transdermal provides certain advantages, the biomaterials must still be introduced into the target location such that the procedure cannot be entirely noninvasive.

There are currently no known arthroscopic 3D printing approaches. The closest previously-described procedures involve transdermal 3D printing in which the targeted biomaterial is injected subcutaneously, and polymerizing light energy is delivered through the skin. The effectiveness of such an approach can be limited, at least in terms of the depth of the target site, due to diffusion and possible non-uniform transmission through the skin and vascular structures. Further, the lack of direct visualization poses a challenge for precise targeting of the radiation and does not lend itself to fabrication of complex structures.

In view of the foregoing, the need remains for a method and device for in vivo printing of biological structures and supports.

### SUMMARY

According to embodiments disclosed herein, a device is provided to facilitate printing or deposition of biomaterials directly at a target site in a live subject during minimally-invasive arthroscopic surgery. The device is preferably used in conjunction with an arthroscope to enable visualization of the printing process and target. Biomaterials that can be utilized for localized printing/deposition include, but are not limited to, gelatin methacrylate (GelMA), thiolated heparin (Hep-SH), hyaluronic acid (HA), glycidyl methacrylate hyaluronic acid (HA-GM), poly (glycerol sebacate) acrylate (PGSA), polyethylene glycol diacrylate (PEGDA), and polyacrylamide (PAA). These materials can be used to fabricate mechanical support structures at a target location and/or as an implant that provides controlled release of biochemicals, e.g., growth factors (GF), to modulate the biochemical environment at the target. The device provides a combination tool for simultaneously depositing biomaterial at the target site within the body during an arthroscopic procedure and delivering the polymerizing radiation (light) directly to the deposited biomaterial to solidify the structure. Upon exposure to the specified wavelength, the biomaterial will be crosslinked to transform it from its initial liquid state to a solid state.

The inventive device provides for 3D printing of biomaterials within the body with the light source and biomaterial deposition source inserted directly into the surgical field under clinically operational conditions. This method of 3D printing is compatible with any biomaterials that are cross-linkable under light exposure, offering a wide range of applications and tunability based on the intended target.

In one aspect of the invention, a device for in vivo 3D bioprinting includes an elongated hollow tube having a distal end and a proximal end, the hollow tube configured for insertion into a living body at a target site; a feed tube housed within the hollow tube, the feed tube configured to convey a liquid polymerizable biomaterial from a biomaterial source disposed near the proximal end to the distal end; an extrusion nozzle disposed at the distal end of the feed tube, the nozzle configured to extrude the biomaterial at the target site; a light guide disposed within the hollow tube, the light guide configured to conduct polymerizing light from a light source to the distal end; and a light transmissive lens disposed at the distal end for directing polymerizing light toward the biomaterial that has been extruded at the target site. In one embodiment, the light transmissive lens has an annular configuration that is concentric with the extrusion nozzle. In some embodiments, the hollow tube may be associated with a viewing arthroscope so that the hollow tube and viewing arthroscope are inserted together in conjunction with an arthroscopic procedure. The biomaterial source may be a container in fluid communication with the feed tube, where a plunger may be used to apply pressure to the biomaterial within the container to force the biomaterial into the feed tube at a controlled rate. A plunger motor may be provided to drive the plunger when activated by a device user. In some embodiments, the biomaterial is one or more material selected from gelatin methacrylate (GelMA), poly (glycerol sebacate) acrylate (PGSA), hyaluronic acid (HA), glycidyl methacrylate HA (HA-GM), polyacrylamide (PAA), biocompatible hydrogels, and polyethylene glycol diacrylate (PEGDA). The biomaterial may further include one or more of thiolated heparin (Hep-SH) and a growth factor (GF).

In another aspect of the invention, a method for in vivo 3D bioprinting includes inserting the distal end of an elongated hollow tube into a living body at a target site; conveying a liquid polymerizable biomaterial from a biomaterial source through a feed tube disposed within the hollow tube to an extrusion nozzle disposed at the distal end of the feed tube, the nozzle configured to extrude the biomaterial at the target site; delivering polymerizing light to the distal end through a light guide disposed within the hollow tube to a light transmissive lens and directing polymerizing light toward the biomaterial that has been extruded at the target site to solidify the biomaterial. In some embodiments, the method may further include repeating the steps of feeding and delivering polymerizing light to construct multiple layers of biomaterial. In some embodiments, at least one layer of the multiple layers may have a different composition than one or more other layer. The step of inserting may include associating the hollow tube with a viewing arthroscope so that the hollow tube and viewing arthroscope are inserted together. The biomaterial source may be a container in fluid communication with the feed tube, where a plunger may be used to apply pressure to the biomaterial within the container to force the biomaterial into the feed tube at a controlled rate. A plunger motor may be provided to drive the plunger when activated by a device user. In some embodiments, the biomaterial is one or more material selected from gelatin methacrylate (GelMA), poly (glycerol sebacate) acrylate (PGSA), hyaluronic acid (HA), glycidyl methacrylate HA (HA-GM), polyacrylamide (PAA), biocompatible hydrogels, and polyethylene glycol diacrylate (PEGDA). The biomaterial may further include one or more of thiolated heparin (Hep-SH) and a growth factor (GF).

### BRIEF DESCRIPTION OF THE DRAWINGS

FIG. 1A is an exploded perspective view of an embodiment of a device for delivering a liquid polymerizable biomaterial; FIG. 1B illustrates an exemplary use of an embodiment of the inventive delivery device for localized in vivo printing to secure a shoulder tendon to a humerus during arthroscopic surgery.

FIG. 2 is a cross-sectional view of an optical path according to the embodiment of FIG. 1A.

FIG. 3 is a detail perspective view of an exemplary delivery tip of the embodiment of FIG. 1A.

FIG. 4 is a plot of changes in effective Young's modulus with different light intensities.

FIGS. 5A-5B plot tensile modulus and ultimate tensile strength, respectively, with variations in exposure and composition of the biomaterials in a double network structure.

### DETAILED DESCRIPTION OF EMBODIMENTS

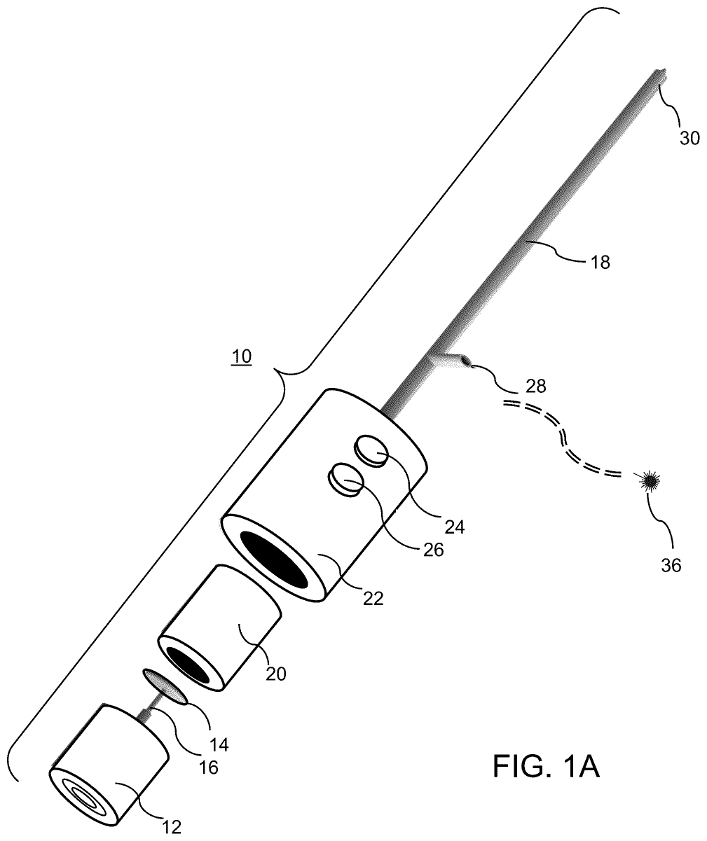

Referring to FIGS. 1A-3, the combined delivery and exposure device includes an extrusion tip for introducing the biomaterial, also referred to as “bioink,” to a target site in a subject's body, and a polymerizing light source to induce crosslinking of the selected biomaterial at the target site. In one embodiment, a UV light source with a wavelength within a range of ˜380 nm-˜410 nm may be used, with selection of the appropriate wavelength and other energy parameters being based on the specific biomaterial(s) used and the application.

The inventive scheme employs general principles of 3D bioprinters that are known in the art. See, for example, the 3D bioprinters disclosed in U.S. Pat. Nos. 10,464,307, 9,361,171, and 11,440,225, each of which is incorporated herein by reference. Briefly, in such printers, printing is achieved by exposing a pre-polymer solution to polymerizing light modulated by a series of patterned masks to progressively form structures. According to the inventive approach, rather than projecting modulated light onto a printing platform or surface that supports a container of pre-polymer solution, the biomaterial is extruded through delivery assembly 10, a sample implementation of which shown in FIG. 1A. Assembly 10 includes a long, thin tube 18 that has a distal end 30 configured to be inserted through a surgical incision or through a body opening of a patient, as shown in FIG. 1B. Tube 18, formed from medical grade stainless steel, or a rigid polymer amenable to standard medical sterilization procedures, has a co-axial feed tube 32 formed therein to define optical channel 21 between the inner wall of tube 18 and the outer surface of tube 32. The dimensions of assembly 10 fall within the general dimensions of a typical arthroscope: the outer diameter of tube 18 may be on the order of about 2.5 to 6 mm with an overall length of about 100 to 190 mm. Selection of an appropriate inner diameter for feed tube 32 will be guided by a combination of the dimensions of tube 18 and the characteristics of the biomaterial to be dispensed. As will be apparent to those in the art, use of the device to dispense a biomaterial with relatively high viscosity may support selection of a large inner diameter. Polymerizing light from light source 36 is directed into optical channel 21 by way of a port 28 into the interior of tube 18. Optical channel 21 serves as a light guide, directing the light 42 into and towards distal end 30 to expose and polymerize biomaterial that has been extruded from the end of the tube.

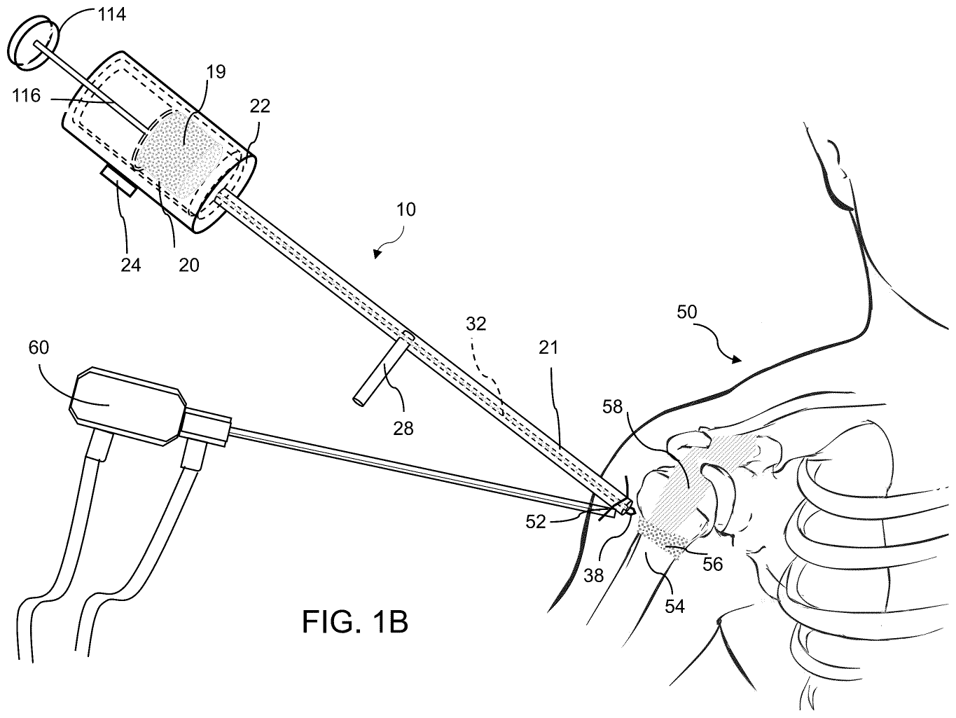

FIG. 2 illustrates details of the optical path of the assembly as well as the internal construction of delivery assembly 10. Light from light source 36 is directed (via a conventional light cable (not shown)) into port 28 which is connected to tube 18, where the light 42 is redirected through optical channel 21 toward the distal end 30 by mirror 38. The illustrated configuration of port 28 as perpendicular to tube 18 is exemplary only. A shallow angle intersection may not require a mirror—the goal is to direct light 42 toward distal end 30. Where a mirror is used, it will typically have an annular configuration to permit co-axial feed tube 32 to pass through its center. In the illustrated example, mirror 38 is arranged at a 45° angle to redirect the incoming light 42 from port 28 at a right angle. As will be apparent to those in the art, the entry angle of the port and the angle of mirror 38, if used, may be varied to ensure an optical path that is coincident with the axis of tube 18. One or both of the inner surface of tube 18 and the outer surface of feed tube 32 may optionally be polished or coated for maximal reflection for efficient light transmission through the optical channel 21.

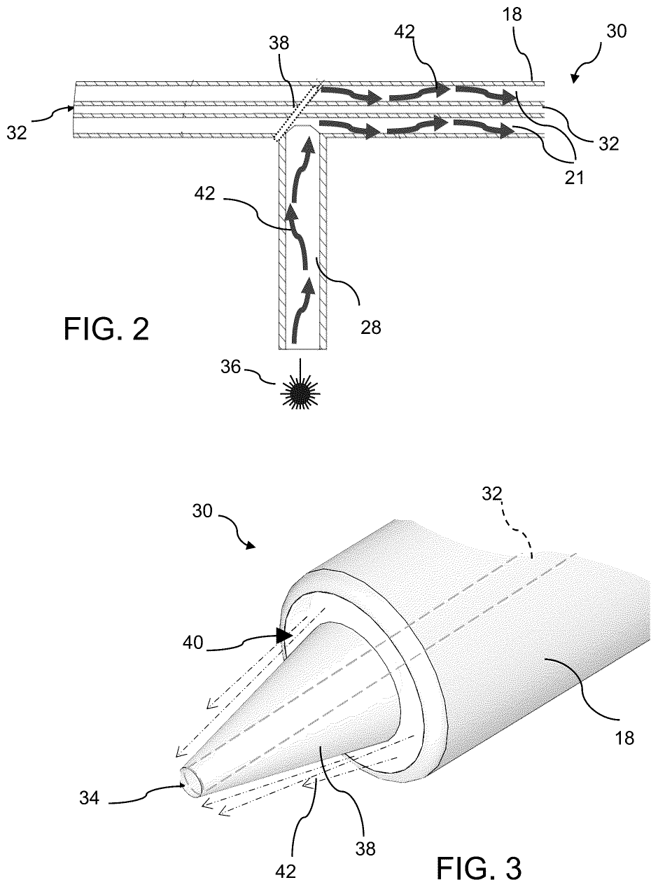

As shown in FIG. 3, at distal end 30, tube 18 terminates at an extrusion nozzle 38 with an annular lens 40 surrounding the nozzle. The lens 40 may be configured to focus, expand, or diffuse the light 42 based on the desired exposure parameters, which may depend on a number of variables including the biomaterial, the wavelength of exposure light, the size and characteristics of the structure to be fabricated, etc. Extrusion nozzle 38 has an opening 34 at its distal end through which biomaterial is dispensed. The shape and length of nozzle tip 38, and the shape and dimensions of opening 34, may vary depending on the biomaterial characteristics, the target site features, and the structure to be fabricated. For example, in some embodiments, opening 34 may be an elongated rectangle or oblong to dispense a ribbon of biomaterial, while in other embodiments, the circular opening (as shown) or an oval dispenses a bead of material.

Referring again to FIG. 1A, the proximal end of tube 18 is attached in fluid connection with handle 22. For simplicity, handle 22 is illustrated as a cylinder, however, the external shape may be tapered and contoured to facilitate handling. Biomaterial container 20 is inserted into a cavity in handle 22 and plate 14 of plunger 16 is placed against the bottom of container 20 to compress the bottom of the container to force biomaterial out of the container and into a feed tube within tube 18. The container 20 may be refillable and reusable or may be a single-use container that is prefilled with the appropriate biomaterial for a particular procedure. In some embodiments, a motor 12 may be used to activate plunger 16 by pressing button 26 (on handle 22), which is electrically connected to motor 12 to switch the motor on and off. In an alternative implementation, an example of which is shown in FIG. 1B, a syringe-like plunger 116 may be used to apply the biomaterial by manually depressing flange 114. A button 24 on handle 22 may be electrically connected (via cables or conductors (not shown)) to light source 36 to activate the light for polymerization of the biomaterial as it is dispensed from nozzle 38 at the target location. As will be readily apparent to those in the art, the user controls may take a variety of different forms. The illustrated buttons in the exemplary embodiments are provided as one possible implementation and are not intended to be limiting.

Still referring to FIG. 1B, during a procedure, the distal end 30 of the device is inserted through an incision 52 at the surgical site. In the illustrated example, the procedure involves a surgical repair to be made to the shoulder of a patient 50. In a typical procedure, the inventive delivery assembly will be used in conjunction with a viewing scope 60 to allow the surgeon to view the procedure. The view scope 60 may be separate from the delivery assembly (as illustrated) or may be physically coupled to the tube 18 to facilitate manipulation During the printing process, the biomaterial is extruded from the nozzle 38 at a user-defined rate, selectable by either pressing button 26 to activate the motor 12 or depressing plunger 116 to introduce the biomaterial 19 from container 20 into the feed tube 32 and out of the nozzle opening 34 to a selected location of the patient's humerus 54. As the biomaterial is extruded from nozzle 38, the user depresses button 24 to activate the polymerizing light source 36. As it exits the tip, the biomaterial 56 will solidify upon exposure to the light that has been guided through the light guide to the lens 40 at the end of the tube 18, allowing the surgeon to effectively “spot weld” tissue. In the illustrated example, the goal of the procedure is stabilization of the supraspinatus tendon 58 at the greater tubercle of the humerus 54.

Following the sample procedure described above, for example, a tendon can be “welded” to the bone. A wide range of different structures can be fabricated and procedures performed using the delivery assembly 10 using one or more biomaterial, in combination or in discrete layers, depending on the objective. The desired structure can be fabricated in a single activation or it can be gradually constructed by a series of activations, i.e., a first extrusion and exposure, followed by a second extrusion and exposure, and so on. In such a sequence, different biomaterials and/or different exposure conditions may be used during each step to modify the features and mechanical characteristics of the resulting structure. The resulting polymerized biomaterials can be designed to have varying physical properties in order to provide mechanical support to repaired tissue and can be designed to slowly elute growth factors, drugs, or other bio-effective materials over an extended period of time, e.g., 30 days or more. The mechanical properties of the printed materials can be controlled by varying the light intensity and exposure duration in order to form softer or more rigid regions.

The device can be incorporated into or otherwise combined with an arthroscope to allow in situ, real time visualization of the printing procedure, providing precise placement and dimensions of the biomaterial at the target site.

The inventive device can be used for a number of different arthroscopic procedures, including, but not limited to, rotator cuff repair, microdiscectomy, cartilage microfracture repair, labrum repair, intervertebral disc repair, bone repair, ligament reconstruction, and more. Many different surgical procedures can be improved by employing the inventive device to fabricate custom-designed mechanical reinforcement(s) within a patient's body to help stabilize the surgical site to promote healing. Additionally, the ability to provide for controlled release of bio-effective materials such as growth factors further enhances the healing process.

### EXAMPLES

The following examples describe different materials and procedures that may be used in conjunction with the described delivery device in arthroscopic procedures. These examples are not intended to be limiting, but merely illustrative of possible applications.

### Example 1: Muscle Regeneration

Volumetric muscle loss (VML) injuries due to trauma, tumor ablation, or other degenerative muscle diseases are debilitating and currently have limited options for self-repair. 3D printing in accordance with the devices and procedures described hereinabove provides for the rapid fabrication of biocompatible scaffolds with custom patterns or simply to replace lost tissue volume. Commonly-used materials chosen are often stiff or brittle, which is not optimal for muscle tissue engineering. In addition, the more successful fabrication approaches have employed cell-based regenerative techniques intended to induce organized muscle regeneration. However, regulatory hurdles and immunogenic concerns associated with cellular tissue-engineering scaffolds have made acellular scaffolds more attractive for biomedical applications in treatment of VML. Poly (glycerol sebacate) (PGS) has been shown to be a highly tunable, biodegradable elastic polymer. PGS is highly elastic and has robust mechanical properties, and is able to maintain its structural integrity in an aqueous environment. Its drawback is that it has a high viscosity and a high glass transition temperature, making it difficult to fabricate with the geometric alignment. To avoid these issues, modification of PGS to make poly (glycerol sebacate) acrylate (PGSA) allows for the precise fabrication of structures with tunable material properties similar to those of skeletal muscle. PGSA has also been shown to be biocompatible with fibroblasts, cardiomyocytes, and vascular endothelial cells (e.g., HUVEC).

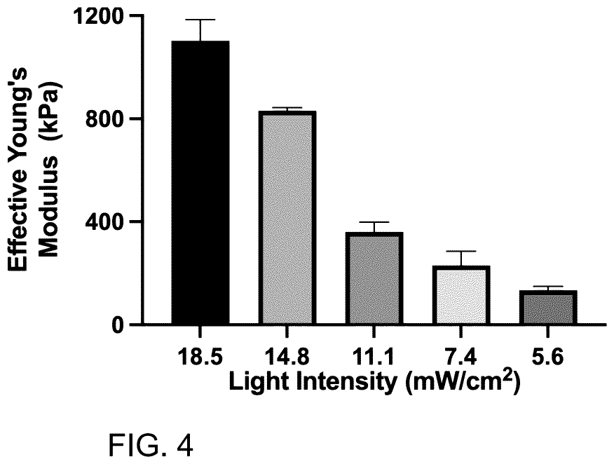

As disclosed in W. Kiratitanaporn, et al. (Biomaterials Advances 142 (2022) 213171), incorporated herein by reference, PGSA printing solutions were prepared and exposed to light at 385 nm. The stiffness of the resulting structure can be varied as a function of light exposure intensity to achieve a stiffness similar to normal skeletal muscle (107 kPa-225 kPA). FIG. 4 provides a plot of effective Young's modulus of PGSA as a function of the light exposure intensity. A light exposure of 5.6 mW/cm² at 385 nm may be used in conjunction with PGSA introduced to the target location via the inventive delivery device, also allowing for the printing of fine structures without overpolymerization.

### Example 2: Controlled Release of Growth Factors

Growth factors (GFs) regulate proliferation and differentiation of cells in order to promote tissue regeneration. GF turnover is rapid in vivo, resulting in short serum half-lives. In order to better stimulate tissue regeneration, tissue engineering strategies often seek to control the release of GFs. In view of their controllable degradability and ability to protect enveloped molecules from degradation, hydrogels can be used to regulate GF release. However, due to high water content, GFs tend to diffuse out quickly from hydrogels since there are no moieties for them to attach.

Due to its high negative charge density, heparin can trap positively charged common proteins, such as GFs, by electrostatic forces, which can be used to prolongate GF release from hydrogels that traditionally are released rapidly from hydrogels. Previous studies have discovered that the kinetics of GF release can be modulated by varying the molecular weight and concentration of heparin in the hydrogel; increased heparin molecular weight and increased heparin concentration result in protracted GF release.

Hyaluronic acid (HA) is a hydrogel which has been widely engineered for applications such as wound healing and atopic dermatitis due to its role in granulation and cell migration. Previous studies have shown that the synthesis of glycidyl methacrylate HA (HA-GM) allows HA to be compatible with light-based 3D printing and provides a mechanism to tune the physical properties and geometry of the hydrogel. The combination of HA and heparin further allows for the ability to modify GF release kinetics over extended periods of time from hydrogels. A thiolated heparin (Hep-SH) can be incorporated into hydrogel structures printed using the techniques described herein to tune GF retention without affecting the mechanical properties of the resulting structure.

To provide for the controlled release of GFs at a target site to promote healing and tissue growth, a multi-material approach can be used in which a bilayer structure of HA-GM and Hep-SH can be formed using serial dispensing and exposure of the biomaterial dispensed for delayed and/or sequential release of multiple GFs. Additional details of the processing and performance of the multilayered structured for controlled release of GFs are provided by P. Wang, et al. (Adv. Healthcare Mater. 2019, 1900977, which is incorporated herein by reference.)

### Example 3: Double Network Structures

Many biomaterials that are used to form structures with complex geometry, for example, polyethylene glycol diacrylate (PEGDA) do not exhibit mechanical properties that appropriately mimic their intended tissue environment. Clinically-used synthetic biomaterials tend to be either too brittle or too soft, limiting their use in more compliant tissues such as skin, vasculature, muscle, and nerve. Tough and elastic biomaterials allow for the development of scaffolds and devices with mechanical properties similar to tissues like skeletal muscle, which routinely goes through cycles of lengthening and shortening, has a specific tension between 125-250 kPa, and undergoes strains up to 40%. Poly (glycerol sebacate) (PGS) has emerged as a tough biomaterial as well as a biodegradable elastomer, however, fabricating complex structures from PGS is challenging due to its high viscosity and glass transition temperature. As a result, most applications incorporating PGS are limited to molding and electrospinning fabrication techniques, which limits their structural complexity for applications such as tissue engineering, where patient-specific designs are of particular importance.

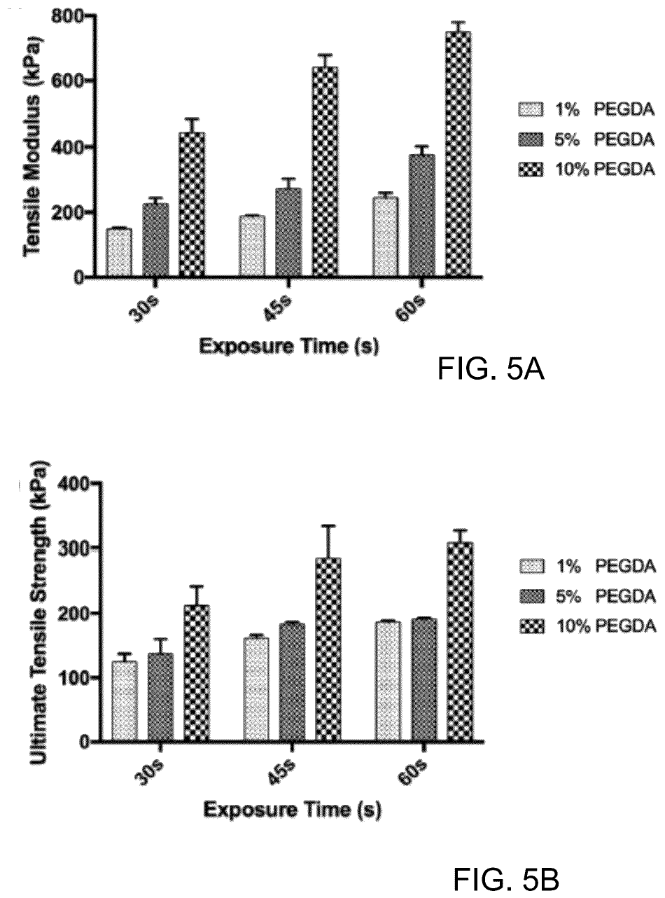

As described in Example 1 above, when PGS is acrylated into PGSA, it becomes more easily tunable for fabrication. A multi-layer structure of PGSA and PEGDA combines the benefits of both materials into a structure in which PGSA enhances the elasticity and PEGDA enhances the mechanical strength of the final structure, in a double network (DN) structure. In addition to varying the composition of the polymer, the mechanical properties of the resulting structure can be tailored by varying the exposure time for printing, which is directly related to the degree of crosslinking. Using light at 405 nm, increasing the exposure time increased the tensile modulus and ultimate tensile strength of the resulting polymer, as shown in FIGS. 5A and 5B. Additional details of the processing and performance of the DN structures are provided by Wang, et al., Adv. Funct. Mater. 2020, 30, 1910391, which is incorporated herein by reference.

The preceding examples illustrate the variety of materials and processing conditions that can be employed in the inventive scheme for in vivo printing. Based on this disclosure, those of skill in the art will recognize that variations in the delivery assembly configuration, the biomaterials and the exposure conditions, and different material dispensing, and exposure sequences, e.g., multiple layers, may be used without deviating from the general principles disclosed herein.

1. A method for in vivo 3D bioprinting to form a structure within a living body in need thereof, the method comprising:
disposing a container with a liquid polymerizable biomaterial into a cavity within a dispensing handle in fluid communication with a proximal end of an elongated dispensing tube;
inserting a distal end of the elongated dispensing tube into an opening in the living body at a target site;
dispensing the biomaterial by applying pressure within the dispensing handle to compress the container and force the biomaterial into the dispensing tube and out an extrusion nozzle disposed at the distal end, the extrusion nozzle configured to extrude the biomaterial at the target site;
activating a polymerizing light at the distal end, the polymerizing light having an exposure intensity and an exposure duration sufficient to polymerize the extruded biomaterial to form the structure having predetermined mechanical properties; and
withdrawing the distal end after the structure is completed.

2. The method of claim 1, further comprising repeating the steps of dispensing and activating to construct a multi-layer structure.

3. The method of claim 2, wherein at least one layer of the multi-layer structure has a different composition than one or more other layer, and further comprising disposing a different container with a different biomaterial in the cavity prior to dispensing.

4. The method of claim 1, wherein the biomaterial is one or more material selected from the group consisting of gelatin methacrylate (GelMA), poly (glycerol sebacate) acrylate (PGSA), hyaluronic acid (HA), glycidyl methacrylate HA (HA-GM), polyacrylamide (PAA), biocompatible hydrogels, and polyethylene glycol diacrylate (PEGDA).

5. The method of claim 2, wherein the multi-layer structure comprises PGSA and PEGDA to define a double network structure having elasticity and strength.

6. The method of claim 5, wherein an amount of PEGDA and the exposure duration are varied to control elasticity and strength of the double network structure.

7. The method of claim 1, wherein applying pressure within the dispensing handle comprises activating a plunger motor configured to drive a plunger against the container.

8. The method of claim 1, wherein the biomaterial further comprises one or more of thiolated heparin (Hep-SH) and a growth factor (GF).

9. The method of claim 1, wherein inserting further comprises associating the dispensing tube with a viewing scope, so that the dispensing tube and viewing scope are inserted together in conjunction with an arthroscopic procedure.

10. The method of claim 1, wherein the structure is configured to attach or stabilize tissue to a bone or other tissue at the target site.

11. The method of claim 1, wherein the structure is configured to replace lost tissue volume at the target site.

12. The method of claim 1, wherein the polymerizing light is within a range of ˜380 nm to ˜410 nm.

13. The method of claim 2, wherein the multi-layer structure comprises HA-GM and Hep-SH configured to provide for controlled release of GFs at the target site to promote healing and tissue growth.

14. The method of claim 1, wherein the biomaterial is PGSA and the light exposure intensity is varied to achieve a stiffness similar to normal skeletal muscle (107 kPa-225 kPA).

15. A method for in vivo 3D bioprinting to form a structure within a living body, the method comprising:
disposing a container with a first liquid polymerizable biomaterial into a cavity within a dispensing handle in fluid communication with a proximal end of an elongated dispensing tube;
inserting a distal end of the elongated dispensing tube into an opening in the living body at a target site;
dispensing the first biomaterial by applying pressure within the dispensing handle to compress the container and force the first biomaterial into the dispensing tube and out an extrusion nozzle disposed at the distal end, the extrusion nozzle configured to extrude the first biomaterial at the target site;
activating a polymerizing light at the distal end, the polymerizing light having an exposure intensity and an exposure duration sufficient to polymerize the extruded first biomaterial to form at least one first layer of the structure having predetermined mechanical properties;
disposing a second container with a second biomaterial into the cavity and repeating the steps of dispensing and activating to form one or more second layer on top of the at least one first layer to construct a multi-layer structure; and
withdrawing the distal end after the multi-layer structure is completed.

16. The method of claim 15, wherein each of the first and second biomaterial is one or more material selected from the group consisting of gelatin methacrylate (GelMA), poly (glycerol sebacate) acrylate (PGSA), hyaluronic acid (HA), glycidyl methacrylate HA (HA-GM), polyacrylamide (PAA), biocompatible hydrogels, and polyethylene glycol diacrylate (PEGDA).

17. The method of claim 15, wherein the multi-layer structure comprises PGSA and PEGDA to define a double network structure having both elasticity and strength.

18. The method of claim 17, wherein an amount of PEGDA and the exposure duration are varied to control elasticity and strength of the double network structure.

19. The method of claim 15, wherein at least one of the first biomaterial and the second biomaterial further comprises one or more of thiolated heparin (Hep-SH) and a growth factor (GF).

20. The method of claim 15, wherein the structure is configured to attach or stabilize tissue to a bone or other tissue at the target site.

21. The method of claim 15, wherein the structure is configured to replace lost tissue volume at the target site.
